(19) United States
(12) Patent Application Publication
(10) Pub. No.: US 2023/0049048 A1
(43) Pub. Date: Feb. 16, 2023

(54) METHODS AND APPARATUS FOR EFFICIENT AND ACCURATE ASSEMBLY OF LONG-READ GENOMIC SEQUENCES

(71) Applicant: Lodo Therapeutics Corporation, New York, NY (US)

(72) Inventors: Zachary CHARLOP-POWERS, Queens, NY (US); Zachary David KURTZ, Farmingdale, NY (US); Bradley Morgan HOVER, New York, NY (US); Steven L. COLLETTI, New York, NY (US)

(21) Appl. No.: 17/797,317

(22) PCT Filed: Feb. 5, 2021
(86) PCT No.: PCT/US2021/016916

§ 371 Date: Aug. 3, 2022

Related U.S. Application Data
(60) Provisional application No. 62/971,394, filed on Feb. 7, 2020.

(51) Int. Cl. G16B 30/00; G16B 40/20

(52) U.S. Cl. CPC G16B 30/00; G16B 40/20

(57) ABSTRACT

The present application generally relates to identifying gene clusters from long-read genomic sequencing data. The disclosure provides methods, non-transitory computer readable media, and apparatuses for processing long-read genomic sequencing data, performing error corrections, and identifying gene cluster, e.g. biosynthetic gene clusters. The methods, non-transitory computer readable media, and apparatuses described herein can be employed in broad areas of biological applications, such as drug discovery, industrial chemical discovery and production, and basic biological research.

### CROSS REFERENCE TO RELATED APPLICATIONS

This application claims the benefit of priority to U.S. Provisional Patent Application Ser. No. 62/971,394, filed on Feb. 7, 2020 and titled “Methods and Apparatus for Efficient and Accurate Assembly of Long-Read Genomic Sequences”, the contents of which are hereby incorporated by reference in their entireties.

### BACKGROUND

Genome sequencing technologies have advanced through several generations, resulting in lower sequencing costs and higher sequencing throughput. Some known long-read sequencing technologies allow for rapid sequencing of genomes within microbial communities found in the environment. Some existing long-read sequencing methods such as single molecule real-time sequencing and nanopore sequencing are currently capable of reading polynucleotide lengths greater than 10 kb and, in some cases, have no theoretical upper limit of read length but have high error rates, ranging from 10-20%. The high error rates lead to “noisy” long-read sequencing data and represent a major challenge in the field for analyzing the sequencing data, including genome assembly and correcting read errors. There is a need for methods that improve the efficiency of genome assembly and error correction in long-read sequencing data derived from genomes within microbial communities, and that can be useful in numerous applications including, for example, identification of gene clusters.

### SUMMARY

The present application generally relates to processing long-read sequencing data for the identification of gene clusters, e.g. biosynthetic gene clusters.

In one embodiment, a method of identifying biosynthetic gene clusters from long-read sequencing data includes: obtaining long-read sequencing data including a set of reads derived from a sample of genomic deoxyribonucleic acid (DNA), each read from the set of reads includes a polynucleotide sequence; partitioning each read from the set of reads into a group of reads from a set of groups, the polynucleotide sequence for each read within each group of reads from the set of groups having a higher alignment length with the polynucleotide sequence for each remaining read in that group of reads than with the polynucleotide sequence for each read within each remaining group of reads from the set of groups; performing a first read error correction for each group of reads from the set of groups by generating a consensus sequence associated with that group of reads; performing a second read error correction for each group of reads from the set of groups by aligning the consensus sequence for that group of reads with a polynucleotide sequence encoding a polypeptide, wherein the consensus sequence is modified to encode the polypeptide, thereby generating a modified consensus polynucleotide sequence; classifying the modified consensus polynucleotide sequence for each group of reads from the set of groups using a machine learning classifier trained to classify polynucleotide sequences belonging to a biosynthetic gene cluster according to a set of polynucleotide sequence features, the classifying including identifying the modified consensus sequence for each group of reads from the set of groups as having the features of the biosynthetic gene cluster; and expressing the modified consensus polynucleotide sequence in a host cell based on identifying the modified consensus sequence as having the features of the biosynthetic gene cluster. In some implementations, the polynucleotide sequence of each read within each group of reads has an alignment length of at least 90%, at least 95%, at least 98%, or at least 99% with the polynucleotide sequence of each remaining read in that group of reads.

In some implementations, the long-read sequencing data is obtained from a database. In some implementations, the long-read sequencing data is obtained by sequencing a sample of genomic DNA using a long-read sequencing method. In some implementations, the sample of genomic DNA is digested into fragments, and wherein the fragments are cloned into a genomic DNA library prior to sequencing. In some implementations, the genomic DNA library includes cosmid vectors.

In some implementations, the machine learning classifier is trained using a set of training data including features extracted from polynucleotide sequences encoding biosynthetic gene clusters and associated classifications, wherein the set of training data is retrieved from a database. In some implementations, the features are selected from open-reading-frames, protein-domain content of open reading frames, promoter binding sites, substrate specificity prediction of enzymatic open reading frames, active site prediction of enzymatic open reading frames. In some implementations, the classifications are selected from structural, chemical, phenotypic, or biosynthetic higher order categories. In some implementations, the machine learning classifier includes at least one of a neural network, a decision tree, a random forest, a support vector machine, a gradient boosting tree, a Bayesian network, or a genetic algorithm.

In one embodiment, a non-transitory processor-readable medium storing code representing instructions to be executed by a processor, the instructions include code to cause the processor to: obtain long-read sequencing data including a set of reads derived from a sample of genomic deoxyribonucleic acid (DNA), each read from the set of reads includes a polynucleotide sequence; determine an alignment length between the polynucleotide sequence of each read from the set of reads and the polynucleotide sequence of the remaining reads from the set of reads; generate a network graph representation of the set of reads and the alignment length for each read from the set of reads, each node from a set of nodes in the network graph representation represents a single read from the set of reads and each edge from a set of edges between a pair of nodes from the set of nodes represents that the alignment length between the polynucleotide sequence of a first read from a pair of reads and the polynucleotide sequence of a second read from the pair of reads is above an alignment length threshold; partition each read from the set of reads into a group of reads from a set of groups, the polynucleotide sequence for each read within each group of reads from the set of groups having a higher alignment length with the polynucleotide sequence for each remaining read in that group of reads than with the polynucleotide sequence for each read within each remaining group of reads from the set of groups; generate a consensus polynucleotide sequence for each group of reads from the set of groups based on the polynucleotide sequence associated with each read from the set of reads in that group; align the consensus polynucleotide sequence for each group of reads from the set of groups with a polynucleotide sequence encoding a polypeptide, wherein the consensus polynucleotide sequence for that group of reads is modified to encode the polypeptide to produce a modified consensus polynucleotide sequence; classify the modified consensus polynucleotide sequence for each group of reads from the set of groups using a machine learning classifier trained to classify polynucleotide sequences belonging to a biosynthetic gene cluster according to a set of polynucleotide sequence features to identify polynucleotide sequences belonging to one or more biosynthetic gene clusters from long-read sequencing data. In some implementations, the alignment length threshold is at least 90%, at least 95%, at least 98%, at least 99%, or 100% alignment length. In some implementations, the polynucleotide sequence encoding a polypeptide is from a database of biosynthetic gene clusters.

In one embodiment, an apparatus includes: a memory; a communicator; and a processor operatively coupled to the memory and the communicator, the processor configured to: obtain long-read sequencing data including a set of reads derived from a sample of genomic deoxyribonucleic acid (DNA), each read from the set of reads includes a polynucleotide sequence; determine an alignment length between the polynucleotide sequence of each read from the set of reads and the polynucleotide sequence of the remaining reads from the set of reads; generate a network graph representation of the set of reads and the polynucleotide alignment lengths for each read from the set of reads, each node from a set of nodes in the network graph representation represents a single read from the set of reads and each edge from a set of edges between a pair of nodes from the set of nodes represents an alignment length between the polynucleotide sequence of a first read from a pair of reads and the polynucleotide sequence of a second read from the pair of reads is above an alignment length threshold of the length of either of the reads; partition each read from the set of reads into a group of reads from a set of groups, the polynucleotide sequence for each read within each group of reads from the set of groups having a higher alignment length with the polynucleotide sequence for each remaining read in that group of reads than with the polynucleotide sequence for each read within each remaining group of reads from the set of groups; generate a consensus polynucleotide sequence for each group from the set of groups based on the polynucleotide sequence associated with each read from the set of reads in that group; align the consensus polynucleotide sequence for each group of reads from the set of groups with a polynucleotide sequence encoding a polypeptide based on the polynucleotide sequence encoding the polypeptide, wherein the consensus polynucleotide sequence for that group of reads is modified to encode the polypeptide, thereby producing a modified consensus polynucleotide sequence; classify the modified consensus polynucleotide sequence for each group of reads from the set of groups using a machine learning classifier trained to classify polynucleotide sequences belonging to a biosynthetic gene cluster according to a set of polynucleotide sequence features; and output a report identifying polynucleotide sequences belonging to the biosynthetic gene cluster. In some implementations, the alignment length threshold is at least 90%, at least 95%, at least 98%, at least 99%, or 100% alignment length. In some implementations, the polynucleotide sequence encoding a polypeptide is from a database of biosynthetic gene clusters.

### BRIEF DESCRIPTION OF THE DRAWINGS

FIG. 1 is a diagram depicting a workflow for generating annotated gene clusters from long-read genomic sequencing data, according to an embodiment.

FIG. 2 is a diagram depicting an apparatus for identifying and classifying gene clusters, according to an embodiment.

FIG. 3A is a diagram depicting a workflow for partitioning long-read sequence data into groups using sequence similarity and/or sequence alignment as a molecular identifier and performing error correction using consensus sequences, according to an embodiment.

FIG. 3B is a diagram depicting a workflow for partitioning long-read sequence data into groups using sequence similarity and/or alignment as a molecular identifier and performing error correction using consensus sequences, according to an embodiment.

FIG. 4 is a diagram depicting an error correction process of genomic sequence data that uses both consensus sequences and frameshift corrections, according to an embodiment.

FIG. 5 is a graph showing the improvement of frameshift-based error correction on assembled genomic sequence data, according to an embodiment.

FIG. 6 is a graph showing the effectiveness of the frameshift error correction as a function of frameshift penalty using either protein identity (Genes) or domain-content (Domains) to assess similarity, according to an embodiment.

FIG. 7 is a graph showing the effect of the number of reads on the accuracy of assembling two reference populations, according to an embodiment.

FIG. 8 is a diagram depicting nucleotides extracted from soil being sequenced, annotated, and deposited into a database, according to an embodiment.

FIG. 9 is a diagram depicting a workflow for predicting cyclic or linear peptide structure from nucleotide sequence data, according to an embodiment.

FIG. 10 is a diagram showing the results of machine learning model classification and scoring of peptide structural features from nucleotide sequences, according to an embodiment.

FIG. 11 is a graph showing accuracy of a machine learning model for predicting peptide structural features from genomic features derived from error-corrected nucleotide sequences, according to an embodiment.

FIG. 12 is a graph showing ranking of importance of genomic features derived from nucleotide sequences in predicting structural features of the encoded compound as predicted by a trained machine learning model, according to an embodiment.

FIG. 13 is a series of plots demonstrating the reproducibility, statistical error for detection of contigs and contigs biosynthetic gene clusters detected by the informatics pipeline within runs and between runs for a library of long-read sequencing data.

FIG. 14 is a table summarizing the results for 15 separate analyses performed by the bioinformatics pipeline on libraries of long-read sequencing data.

FIG. 15 is a table showing the class of biosynthetic gene clusters identified by the informatics pipeline described by the methods and apparatus of the disclosure. Chemical classes represented in the table include non-ribosomal peptides (NRP), polyketides, ribosomally synthesized and post-translationally modified peptides (RiPP), saccharides, terpenes, or another class of gene cluster (other).

### DETAILED DESCRIPTION

Methods and apparatus described herein generally relate to characterizing long-read sequencing data generated from nucleic acid molecules derived from a genomic sample, e.g. an environmental sample. In some implementations, the methods and apparatus may be used in the identification of gene clusters and associated discovery of bioactive natural products. The steps of an example method include physical manipulation of biological material and computer implemented procedures. The physical manipulation steps of such a method can be engineered to generate data compatible specifically with the computer implemented steps.

The physical manipulation steps of an example method are carried out by a human and include, for example, processing of samples using techniques generally related to biochemical and molecular biological techniques. Compared to alternative physical manipulation steps not included in this disclosure, the physical manipulation procedures described herein can, for example, result in computer readable data that is better suited for the computer implemented steps described herein.

The physical manipulation steps can be, for example, collecting an environmental sample, extracting biological material from the sample, processing biological material from the sample to obtain isolated nucleic acid molecules of interest in a form suitable for nucleotide sequencing, and/or sequencing the nucleic acid molecules.

“Nucleic acid molecules” can include, for example, polynucleotides derived from an organism, genomic deoxyribonucleic acid molecules (DNA), and/or ribonucleic acid molecules (RNA). A nucleic acid molecule can, for example, be extracted and isolated from an environmental sample. Nucleic acid molecules may also be referred to as polynucleotides, which can encompass any of the nucleic acid molecules described herein.

“Protein” or “polypeptide” are used herein interchangeably and refer to a sequence of amino acids joined by peptide bonds. Polypeptides can be enzymes, including the enzymes that make up gene clusters, for example, biosynthetic gene clusters.

“Cloning” can be, for example, inserting any nucleic acid molecule into one or more polynucleotide vectors. Polynucleotide vectors with inserted nucleic acid molecules can be, for example, designed to transfect or transduce a microorganism and/or propagate within the microorganism and/or express the inserted nucleic molecule and/or express another gene located on the vector. Cloning can be, for example, a step in generating a DNA library.

A “DNA library” can be, for example, a set of nucleic acid molecules in a form suitable for storage, propagation, and/or sequencing. A DNA library can comprise genomic DNA or fragmented genomic DNA or digested genomic DNA. Genomic DNA can be digested into fragments of a particular size measured in bases. For example, in some implementations of the methods described herein genomic DNA can be digested into 10 kb, 20 kb, 30 kb, 40 kb, 50 kb, 60 kb, 70 kb, 80 kb, 90 kb, 100 kb fragments, or any other length suitable for cloning into a DNA library.

“Sequencing” can be, for example, the process of determining the order of nitrogenous bases in a nucleic acid molecule. Methods for sequencing can include, but are not limited to, nanopore sequencing, single molecule real time sequencing, pyrosequencing and/or shotgun sequencing.

A “read” can be, for example, the output of the sequencing process. Read data can be, for example, digitally stored representations of the order of nitrogenous bases (“base call”) in a sequenced nucleic acid molecule (“sequence”). Reads can include, for example, polynucleotide sequences. Digitally stored read data can be, for example, stored as a computer readable file. Computer readable files containing read data can serve, for example, as the input data for computer implemented steps of an example method.

By “contig” is meant a contiguous segment of the genome made by joining overlapping clones or sequences. A clone contig consists of a group of cloned (copied) pieces of DNA representing overlapping regions of a particular set of sequences derived from long-read sequencing data. A sequence contig is an extended sequence created by merging primary sequences, e.g. polynucleotide sequences derived from long-read sequencing reads, that overlap.

The computer-implemented steps of an example method are generally related to bioinformatics and machine learning techniques.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared.

A “sequence alignment” or “alignment” is a computer implemented informatics technique that can be, for example, the process of aligning individual polynucleotide sequences according to an arbitrary numbering or positioning scheme for the purpose of comparing base calls. Methods of sequence alignment for comparison and determination of percent sequence identity is well known in the art. Optimal alignment of sequences for comparison can be conducted, e.g., by the homology alignment algorithm of Needleman and Wunsch, J. Mol. Biol. 48:443 (1970), by the search for similarity method of Pearson and Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), by manual alignment and visual inspection (see, e.g., Brent et al., Current Protocols in Molecular Biology (2003)), by use of algorithms know in the art including the BLAST and BLAST 2.0 algorithms, which are described in Altschul et al., Nuc. Acids Res. 25:3389-3402 (1977); and Altschul et al., J. Mol. Biol. 215:403-410 (1990), respectively. Software for performing BLAST analyses is publicly available through the National Center for Biotechnology Information.

An “alignment length” is the length over which two polynucleotide sequences align. The alignment length can be expressed, for example, in the number of basepairs in an alignment, or as a percent of the length of a polynucleotide sequence that is aligned. A higher alignment length is a relative comparison between two alignment lengths. For example, an alignment length of 80% between two polynucleotide is a higher alignment length than an alignment length of 60% between two polynucleotide sequences.

A “consensus sequence” is generated using a computer implemented bioinformatics technique that can be, for example, a sequence containing the most frequently represented base call at each position of one or more aligned individual reads.

A “similarity score” can be generated using a computer implemented bioinformatics technique that can be, for example, a value representative of the similarity of two sequences or reads. The value can be, for example, based on the percentage of identical base calls between aligned sequences or reads or a scoring matrix (e.g. a distance matrix).

A “partition”, also referred to as a grouping, cluster, or bin, can be a set of reads or sequences determined using a computer implemented technique. Partitions can be defined by, for example, a unique molecular identifier, for example an alignment length or similarity scores, between reads.

“Genome assembly” or “assembly” can be, for example, an accurate determination of genomic sequences from sequence read data. Assembly of genomic sequences can be performed using a computer implemented bioinformatics method.

“Features” that can be extracted from polynucleotide sequences can be, for example, codon sequences, protein-encoding regions (open-reading frames), and/or the subfeatures of the encoded proteins. Subfeatures of encoded proteins can be, for example, conserved structural protein features like structural or enzymatic domains.

A “wrapper” can be, for example, a script, program or other software that automates the transmittal of an output or input file to or from a computer readable storage device. A wrapper can be, for example, a script that automates the transmittal of an output or input file to or from a processor configured to perform a bioinformatics, machine learning, or other computer implemented process. A wrapper can be, for example, a script that contains parameters or settings used to execute a bioinformatics, machine learning, or other computer implemented process.

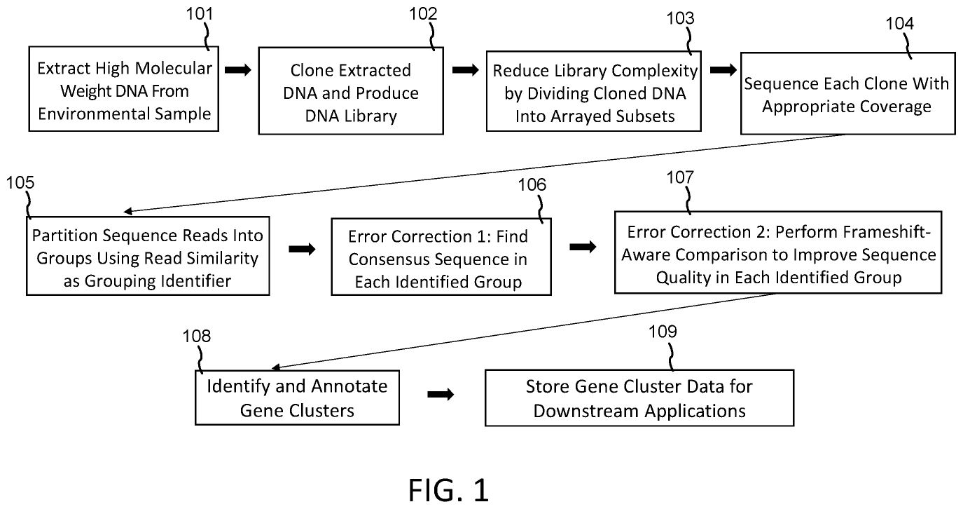

FIG. 1 is a schematic diagram of an example method for producing annotated gene clusters from long-read genomic sequencing data derived from high molecular weight nucleic acids molecules, such as deoxyribonucleic acid (DNA), derived from an environmental sample, according to an embodiment. The method includes, at step 101, extracting DNA from an environmental sample; at step 102, cloning extracted DNA into a DNA vector and transfecting or transducing cloned DNA into a microorganism to create and/or define a DNA library; at step 103, reducing DNA library complexity by dividing the DNA library into arrayed subsets; at step 104, sequencing each clone with an appropriate level of sequencing coverage; at step 105, partitioning sequence reads into groups using a predetermined read similarity and/or alignment length threshold as a grouping identifier; at step 106, performing a first error correction by finding a consensus sequence in each grouping established or defined in step 105; at step 107, performing a second error correction using frameshift-aware comparison to improve sequence quality in each group; at step 108, identifying and annotating gene clusters in each group; and at step 109, storing gene cluster information for downstream applications.

In some embodiments, at step 101, the method of FIG. 1 derives an initial set of nucleic acid molecules from organisms in an environmental sample. The environmental sample can include, for example, samples taken from naturally occurring sources such as, for example, soil, compost, fresh water, salt water, brackish water, air, or another inert media that can host a microorganism. The environment from which a sample is taken can include other environments where microorganisms are found, such as samples taken from the microbiome of plants, animals, insects, humans, or another living organism that hosts a microbiota. The nucleic acid molecules derived from the environmental sample may originate from unicellular or multicellular organisms, including, for example, bacteria, protozoa, fungi, archaea, or algae. Isolation of the organisms can include, for example, processes that filter, separate, or enrich the organisms containing the target nucleic acid molecules from inert or unwanted material, including, for example, unwanted nucleic acid containing matter, from the environmental sample. Such an organism isolation process can include using various laboratory methods (e.g., centrifugation, dilution, vacuum filtration, etc.) or tools, kits, and/or reagents designed for the specific filtration, separation, or enrichment task. Organisms resulting from such steps can be propagated in artificial conditions (e.g., a solid or a liquid culture) prior to extraction of the target nucleic acid molecules. Similar to the organism isolation process, extraction of the nucleic acid molecules can be performed in a number of ways and can include using various laboratory methods (e.g., centrifugation, dilution, vacuum filtration, etc.) or tools, kits, and/or reagents designed for the specific nucleic acid molecule extraction task. In some implementations, step 101 of the method isolates bacteria from a soil sample and extracts target nucleic acid molecules from said bacteria.

In some embodiments, at step 102, the method clones the extracted DNA and produces a DNA library. In some instances, the library is produced from an initial set of nucleic acid molecules isolated from organisms in an environmental sample, as described with respect to step 101. In preparation for production of the DNA library, isolated nucleic acid molecules can be processed by adjusting their length through mechanical, chemical, or biochemical methods. The nucleic acid molecules can be inserted into nucleic acid carriers or vectors, such as, for example, plasmids or cosmids, that include at least one restriction site, a promoter, an origin of replication, a Cos gene, a selectable marker, and/or an antibiotic resistance gene. Insertion of the nucleic acid molecules into the vector can include modifying the ends of the nucleic acid molecules and cutting the vector at specific sites through restriction enzyme digestion and ligating the nucleic acid molecule to the exposed ends of the vector. The vector(s) containing the isolated nucleic acid molecule(s) derived from the environmental sample can be transfected or transduced into a propagated microorganism, according to a scheme that results in a target number of transfected or transduced propagated microorganisms. The transfected or transduced propagated microorganisms make up the DNA library. In some instances, the vector(s) containing the isolated nucleic acid molecule(s) can be extracted from the microorganism and purified.

In some implementations, at step 102, the method produces a DNA library from an initial set of nucleic acid molecules isolated from soil. The isolated nucleic acid molecules are extracted from the soil and inserted into a cosmid vector, such as the pWEB vector. The cosmid vector containing the inserted nucleic acid molecules is packaged into a lambda phage virus. The packaged nucleic acid molecules are transduced into E. coli to reach the formation of, for example, about 0.1E7 clones, 0.25E7 clones, 0.5E7 clones, 0.75E7 clones, 1E7 clones, 1.25E7 clones, 1.5E7 clones, 1.75E7 clones, 2E7 clones, 2.25E7 clones, 2.5E7 clones, 3E7 clones or any other suitable number of clones to create and/or define a DNA library of soil-derived nucleic acid molecules. Advantageously, this number of clones can result in replicates of each nucleic acid molecule in the DNA library, and therefore replicates of each nucleic acid molecule sequence in the data generated in step 104, as described in further detail herein. In some implementations, the E. coli containing the DNA library is preserved in a glycerol solution. In some implementations, the replicated soil-derived nucleic acid molecules including the DNA library are extracted from the E. Coli and purified.

In some embodiments, at step 103, the method divides the DNA library into arrayed subsets to reduce the DNA library complexity. In some implementations, the replicated microorganism containing the DNA library is stored in glycerol, divided into subsets by serial diluted, and stored as arrayed subsets. In some implementations, the DNA library is extracted from the microorganism and purified, divided by serial dilution, and stored as arrayed subsets. Advantageously, dividing the DNA library into an arrayed subset reduces the complexity of analyzing the sequencing data generated in step 104, as described in further detail herein.

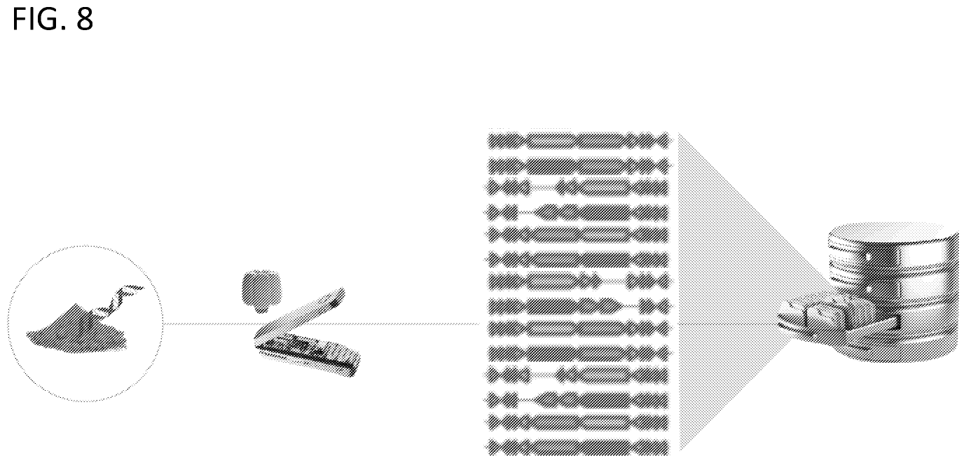

In some embodiments, at step 104, the method sequences the DNA library prepared at step 103. To prepare the DNA library for sequencing, the inserted nucleic acid molecule from each subset of the DNA library can be separated from the vector by restriction enzyme digestion. The separated nucleic acid molecule can be purified from the vector by, for example, low-melt agarose gel electrophoresis, size exclusion chromatography, gel filtration, and/or the like. When, for example, a low-melt agarose gel is used to purify the nucleic acid molecule from the vector, the gel is imaged to identify bands containing the inserted nucleic acid molecule derived from an environmental sample. The bands containing the inserted nucleic acid molecule are cut and/or removed from the gel and the nucleic acid molecule is purified. The purified nucleic acid molecules can be end repaired, and then ligated to sequence adaptors for long-read sequencing, such as, for example, nanopore or single-molecule real time sequencing technologies. Purified nucleic acid molecules modified with sequence adaptors can be sequenced with long-read sequencing technologies at, for example, 2×, 3×, 4×, 7×, 10×, 20×, 38×, or 60× coverage. For example, FIG. 8 shows how the accuracy of a read can improve with increasing coverage. Sequence reads can be output in FASTA, FASTQ, SAM, or other file types used for storing nucleic acid sequence and mapping data.

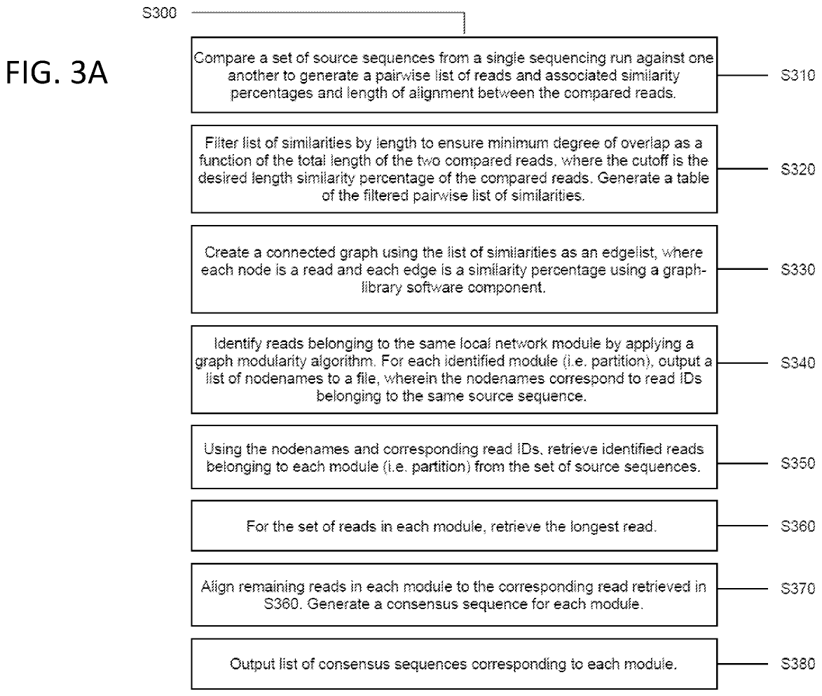

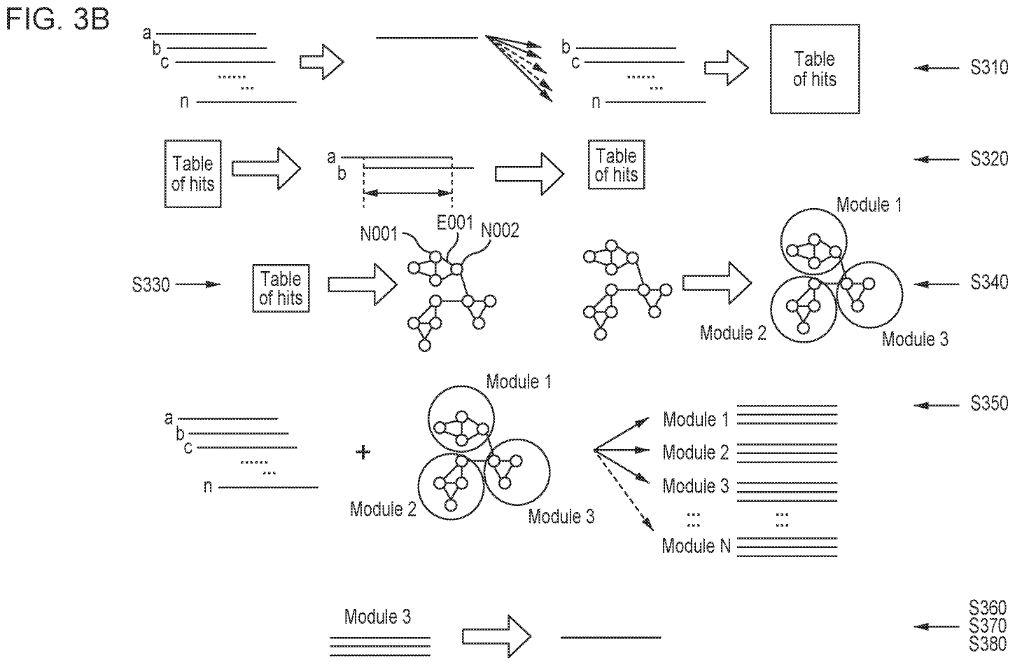

In some implementations, at step 105, the method performs a barcode-free partitioning step of the sequence reads of step 104 using a similarity score and/or alignment length threshold between reads as a unique molecular identifier. Prior to partitioning, contaminating residual vector sequences and sequences below the expected length of a target environmentally-derived nucleic acid molecule, can be filtered out from the total sequence read data. After applying these filters, the remaining sequence reads are originated from environmental nucleic acid molecules and can be compared in an all-against-all fashion to obtain pairwise sequence similarity scores and/or alignment lengths between the reads. For example, FIG. 3A and FIG. 3B, steps S310, S320, S330, S340, and S350 depict the steps for partitioning (binning) a set of reads according to their similarity and/or alignment lengths determined as described herein. According to an embodiment, all-against-all comparison of sequence reads can be carried out using, for example, by importing a FASTA, FASTQ, or SAM file containing said sequence reads into the minimap2 software package. In one implementation, minimap2 locally aligns sequence reads and records nucleotide positions and mismatch properties of sufficiently similar sequences to a file or other storage medium. Comparison scoring of the sequence reads can be a similarity score and/or alignment length. Similarity scores can be, for example, percent sequence similarity, fraction of the sequence read that is locally aligned, or a combination thereof. Alignment length can be, for example, the number of base base pairs over which an alignment between two polynucleotide sequences occurs. Alternatively, an alignment length can be, for example, percent of the total length of a polynucleotide sequences over which an alignment occurs with another polynucleotide sequence. Partitioning the reads based on a similarity score or alignment length can be accomplished by using a graph representation of the all-against-all comparison of sequence reads. The graph contains nodes that can be a representation of individual sequence reads and edges that can be a representation of a pairwise similarity scores and/or alignment length that exceeds a threshold. The threshold similarity score and/or alignment length chosen to generate edges in the network graph can be at least, for example, 5%, 10%, 20%, 30%, 40%, 50%, 60%, 70%, 80%, 90%, 95%, and/or any other suitable percentage. In some implementations, the graph can be generated by, for example, importing the all-against-all comparison output by the minimap2 software package into a network analysis software package, such as, for example, igraph, Gephi, or NetworkX software packages. Nodes in the graph can be partitioned via a score-weighted or unweighted method for community detection, for example, optimal modularity, label propagation, leading eigenvector, or random walk. In one implementation, the Louvain algorithm hierarchically partitions nodes into optimal modules based on the density of edges in the partitioning scheme. In one implementation, the Leiden algorithm partitions nodes into optimal modules based on the density of edges in the partitioning scheme. Each partition can be an ensemble of reads that collectively represent a single nucleic acid molecule derived from the environmental sample. Partitioned reads can be exported in multiple output file formats including, for example, FASTA, FASTQ, and/or SAM for further analysis. Handling of input and output files between data handling steps can be automated by implementing a wrapper script. For example,

In some embodiments, at step 106, the method performs a first error correction step on partitioned sequence reads exported in step 105. Consensus sequences can be generated for the ensemble of sequence reads in each partition. For example, FIG. 3A and FIG. 3B, steps S360, S370, and S380 depict the process for performing a first error correction on the partitioned sets of reads determined in step 105 of the method and generating a consensus sequence. Generating the consensus sequence acts as a method of removing erroneous nucleic acids at a given position in the sequence. Proper sequence alignment of the reads in the partition can be determined by identifying overlapping regions with absolute similarity. Following sequence alignment, the base calls at each position along the length of the sequence are ranked in order of representation at the given position. The most represented base call at each position can be used to generate a new sequence, the consensus sequence, that represents an error corrected sequence of the nucleic acid molecule derived from an environmental sample. The consensus sequence may be generated by, for example, importing each partitioned sequence from step 105 into the Canu or Racon software packages. Receiving the exported, partitioned sequences from step 105 as a FASTA, FASTQ, and/or SAM file type and inputting the file into the consensus sequence generator function of step 106 can be automated by implementing a wrapper script.

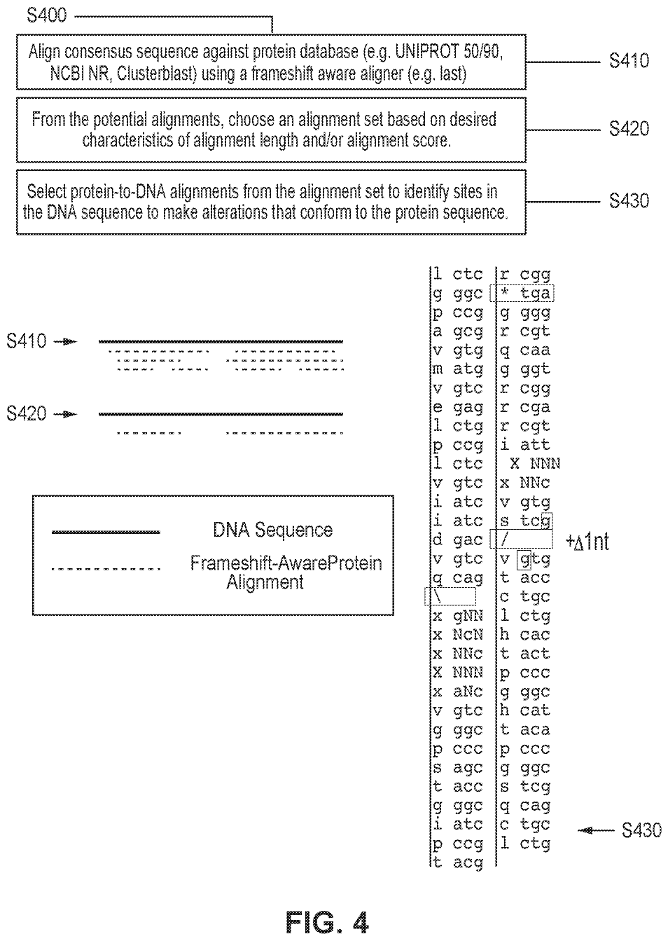

In some embodiments, at step 107, the method performs a second error correction step on consensus sequences exported from step 106, or from another process that generates consensus or assembled sequences from a genome or a metagenome. For example, FIG. 4 steps S410, S420, and S430 depict a workflow for correcting consensus sequences derived from partitioned reads by using frameshift-aware polypeptide-to-polynucleotide alignment. Insertions and deletions are removed from the consensus sequences of step 106 using a frameshift-aware, polypeptide-to-polynucleotide aligner. Each consensus sequence can be aligned to a known, corresponding translated polypeptide sequence. The polypeptide sequences can be retrieved from a database such as Clusterblast, UNIPROT 50/90, NCBI NR.

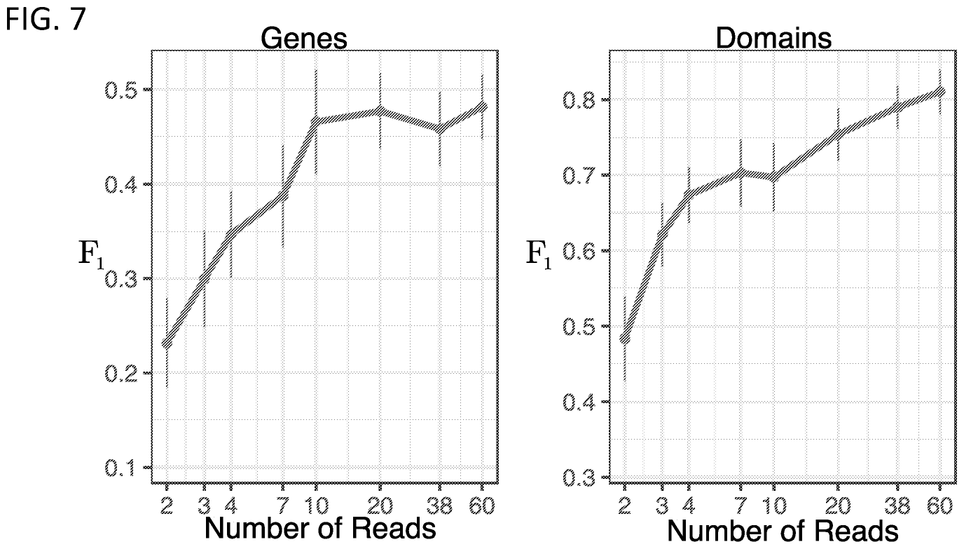

Polynucleotide query sequences are aligned to protein reference sequences using a frameshift penalty (i.e., adding a frameshift cost to an alignment cost function). Frameshift penalties can be, for example, 0, 8, 15, 28, 40, 60, or 100. The frameshift penalty can be used to determine when to allow a nucleotide gap, insertion, and/or deletion. At suitable frameshift penalties (x-axis), the ability to recover features from a reference set can be measured by the F₁ score. A desired F₁ score can be reached by using, for example, recovery of identical protein content or recovery of sub-protein (protein domain) content (FIG. 7). A new sequence is generated, whereby the new sequence contains nucleotide triplets corresponding to confirmed codons and removes a nucleotide when a plus one frameshift is detected, completes known codons by shifting −1 nucleotides to a +1 position, and removes gaps, as exemplified in FIG. 4 at S430. The frameshift-aware error corrected sequences can be generated by, for example, importing a file containing the consensus sequence(s) of step 106 to the lastal software package. Receiving the consensus sequences exported from step 106 as a FASTA, FASTQ, or SAM file type and inputting the file into the frameshift-aware correction function of step 107 can be automated by implementing a wrapper script.

In some embodiments, at step 108, the method annotates and prioritizes gene clusters from corrected sequence reads generated in step 107. A “gene cluster” can be a gene sequence or sets of gene sequences, together with annotations such as protein coding regions. A reference set of known genes or gene clusters, and their associated features, such as the composition of annotations, can be used to train a machine learning model. The machine learning model can be, for example, a random forest model, neural network, support vector machine, gradient boosting tree, and/or another appropriate machine learning model. Reference gene clusters to train the machine learning model can be obtained from public or private repositories and databases, such as, for example, Minimum Information about a Biosynthetic Gene Cluster (MIBiG), Integrated Microbial Genomes Atlas of Biosynthetic Clusters (IMG-ABC), ClusterMine, ChemSpider, Chemical Entities of Biological Interest (chEBI), Pubchem, NCBI RefSeq and/or other sources of features mapped to gene clusters. The trained machine learning model can take as input and categorize or predict properties of the cluster in accordance with the machine learning model generated from the training data. After a feature set has been generated, the machine learning model can be used for ranking, regression or classification of this gene cluster. Sequences from step 107 can also be categorized based on extracted features, such as, for example statistical, structural, chemical, phenotypic, and/or biosynthetic properties by unsupervised clustering. In some implementations, structural categories can be heterocyclic rings or lipophilicity; chemical categories can be the amino acid, carboxylic acid or aklyloid substrates; phenotypic categories can be host bioassay or bioactivity assessment; biosynthetic categories can be biosynthesis class.

In some embodiments, at step 109, the method stores output files from steps 105-108 in a database, as depicted in FIG. 8. The sequences can be stored in a computer-readable medium such as, for example RAMs, ROMs, flash memory, EEPROMs, optical devices (CD or DVD), hard drives, floppy drives, or any suitable device. Sequences and associated data from steps 105-108 can be stored on the database in, for example, JSON, FASTA, FASTQ, FASTS, SAM, GBK, EMBL, or other commonly used file formats for storing or processing polynucleotide or polypeptide sequences. The stored output files can be used in downstream applications. Downstream applications can be, for example, generation and/or training machine learning models (i.e., classifiers) to predict chemical and/or biochemical aspects of gene clusters and/or the generation of applications using such machine learning models.

Embodiments of the method shown and described with respect to FIG. 1 can be used to improve the efficient assembly and annotation of polynucleotide sequencing data from microorganisms in environmental samples. In a specific example, the method can function to improve the genomic assembly of long-read sequencing data from nucleotide molecules originating from a population of bacteria extracted from a soil sample. A computational system, device, and/or apparatus can be used to perform the steps 105-109 of the method. The computational system, device, and/or apparatus can include sequence comparators, artificial intelligence models, feature extractors, and/or feature classifiers. The computational system, device, and/or apparatus can be implemented using, for example, different programming languages, operating systems, programming styles and techniques, and different device interfaces.

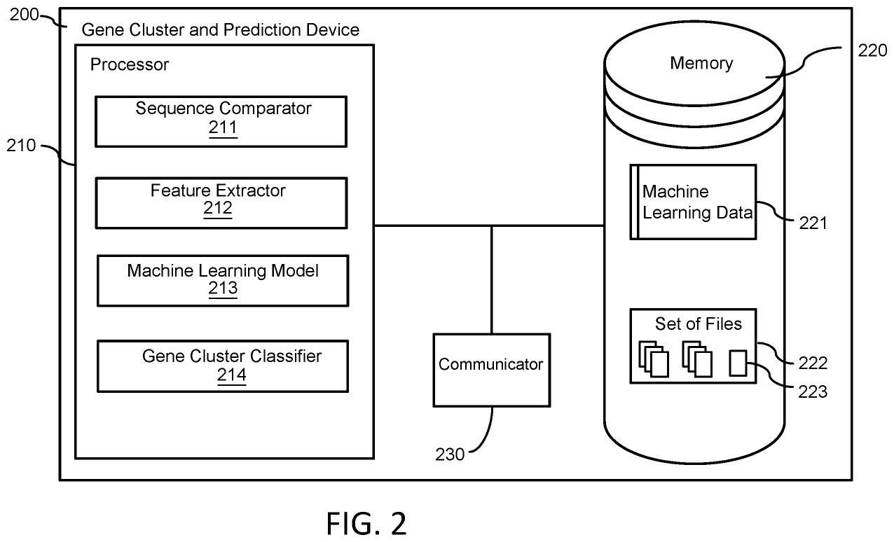

FIG. 2 is a schematic block diagram of a Gene Cluster and Prediction device 200 for predicting properties of a gene cluster from features associated with long-read polynucleotide sequence data derived from environmental samples, according to an embodiment. The Gene Cluster and Prediction device 200, also referred to herein as “the prediction device” or “the device”, can be a hardware-based computing device and/or a multimedia device, such as, for example, a compute device, a server, a desktop compute device, a laptop, a smartphone, a tablet, a wearable device, an implantable device, and/or the like. As described in further detail herein, the device 200 can be used to execute the computer implemented steps 105, 106, 107, 108, and 109 of the example method shown and described with respect to FIG. 1. The device 200 includes a processor 210, a memory 220 and a communicator 230.

In some embodiments, the processor 210 can be, for example, a hardware based integrated circuit (IC) or any other suitable processing device configured to run and/or execute a set of instructions or code. For example, the processor 210 can be a general purpose processor, a central processing unit (CPU), an accelerated processing unit (APU), an application specific integrated circuit (ASIC), a field programmable gate array (FPGA), a programmable logic array (PLA), a complex programmable logic device (CPLD), a programmable logic controller (PLC) and/or the like. The processor 210 is operatively coupled to the memory 220 through a system bus (for example, address bus, data bus and/or control bus).

The memory 220 of the gene cluster and prediction device 200 can be, for example, a random access memory (RAM), a memory buffer, a hard drive, a read-only memory (ROM), an erasable programmable read-only memory (EPROM), and/or the like. The memory 220 can store, for example, one or more software modules and/or code that can include instructions to cause the processor 210 to perform one or more processes, functions, and/or the like (e.g., the sequence comparator 211, feature extractor 212, the machine learning model 213, and/or the gene cluster feature classifier 214). In some implementations, the memory 220 can be a portable memory (e.g., a flash drive, a portable hard disk, and/or the like) that can be operatively coupled to the processor 210. In other instances, the memory can be remotely operatively coupled with the gene cluster and prediction device. For example, a remote database server can be operatively coupled to the gene cluster and prediction device.

The memory 220 can store machine learning data 221 and a set of files 222. The machine learning data 221 can include data generated by the machine learning model 213 during classification of a file (e.g., temporary variables, return addresses, and/or the like). The machine learning data 221 can also include data used by the machine learning model 213 to process and/or analyze a file (e.g., number of trees in a random forest model).

In some instances, the machine learning data 221 can also include data used to train the machine learning model 213. In some instances, the training data can include multiple sets of data. Each set of data can contain at least one pair of an input file and an associated desired output value or label. For example, the training data can include input files that contain the features of polynucleotide sequences, such as open-reading-frames, protein-domain content of open reading frames, promoter binding sites or other features as well as aggregated counts, or composition of features., Additional data can include defined category labels such as, for example, aspects of the predicted compounds such as whether the encoded compound is linear or cyclic, aspects of the chemical itself such as molecule weight, log P, or total surface volume, the molecular formula, or structure of the compound itself. The training data can be used to train the machine learning model 213 to perform classification, ranking, regression or other tasks, given polynucleotide sequence features.

The communicator 230 can be a hardware device operatively coupled to the processor 210 and memory 220 and/or software stored in the memory 220 and executed by the processor 210. The communicator 230 can be, for example, a network interface card (NIC), a Wi-Fi™ module, a Bluetooth® module and/or any other suitable wired and/or wireless communication device. Furthermore, the communicator 230 can include a switch, a router, a hub and/or any other network device. The communicator 230 can be configured to connect the gene cluster and prediction device 200 to a communication network (not shown in FIG. 2). In some instances, the communicator 230 can be configured to connect to a communication network such as, for example, the Internet, an intranet, a local area network (LAN), a wide area network (WAN), a metropolitan area network (MAN), a worldwide interoperability for microwave access network (WiMAX®), an optical fiber (or fiber optic)-based network, a Bluetooth® network, a virtual network, and/or any combination thereof.

In some instances, the communicator 230 can facilitate receiving and/or transmitting a file and/or a set of files through a communication network. In some instances, a received file can be processed by the processor 210 and/or stored in the memory 220 as described in further detail herein.

In some embodiments, the processor 210 can include a sequence comparator 211, a feature extractor 212, a machine learning model 213, and a gene cluster classifier 214. The sequence comparator 211, the feature extractor 212, the machine learning model 213, and/or the gene cluster feature classifier 214 can be software stored in memory 220 and executed by processor 210 (e.g., code to cause the processor 210 to execute the sequence comparator 211, the feature extractor 212, the machine learning model 213, and/or the gene cluster feature classifier 214 can be stored in the memory 220) and/or a hardware-based device such as, for example, an ASIC, an FPGA, a CPLD, a PLA, a PLC and/or the like.

In some embodiments, the sequence comparator 211 can be configured to receive a file as an input. The file can include polynucleotide sequences, polypeptide sequences, and/or features associated with specific sequences. The input file can be any suitable file type such as, for example, JSON, FASTA, FASTQ, FASTS, SAM, GBK, EMBL, or any other file formats used to store and transfer polynucleotide sequences, polypeptide sequences, and/or data associated with specific sequences. Data associated with specific sequences can include statistical features, structural features, chemical features, functional features, relatedness, similarity, and/or alignment length to other sequences (e.g., percent similarity, percent alignment, interaction networks, etc.), relatedness to phenotypes, and/or the like.

In some embodiments, the sequence comparator 211 can perform steps 105-107 of the method shown and described with respect to FIG. 1. Specifically, the sequence comparator 211 can be configured to receive or retrieve as input a file containing at least two polynucleotide or polypeptide sequences and align the two or more sequences for comparison. Alignment and comparison of the two or more sequences can include polynucleotide to polynucleotide comparisons, polynucleotide to polypeptide comparisons, and/or polypeptide to polypeptide comparisons. The sequence comparator 211 can be configured to calculate the similarity and/or alignment length between two or more polynucleotide and/or polypeptide sequences according to the percentage of similarity and/or alignment length of specific nucleotides and/or amino acids at aligned positions. The sequence comparator 211 can be configured to perform a grouping, partitioning, and/or clustering function on sequences based on one or more identifying properties, such as, for example, sequence similarity, alignment length, distance in a network topology, or data associated with specific sequences or groups of sequences. The sequence comparator 211 can be configured to perform error correction on sequences that have been grouped, partitioned, or clustered by determining a consensus sequence within a group, partition, or cluster. The sequence comparator 211 can be configured to perform error correction on a subject polynucleotide sequence by performing an alignment between the subject polynucleotide and an associated polypeptide to identify codon alignments, base pair mismatches, base pair insertions, base pair deletions, and/or gaps in the sequence. Based on the identified errors identified in the polynucleotide and polypeptide alignment and analysis, the polynucleotide sequence can be modified to add, remove, or ignore differences between the polynucleotide and polypeptide sequences. The sequence comparator executes code for processes that retrieve files from memory 220, perform alignment and similarity calculation methods (e.g., basic local alignment search tool (BLAST) and minimap2), partitioning (i.e., grouping or clustering) methods (e.g., igraph and Louvain method for community detection), calculating consensus sequences (e.g., Racon and Canu), software wrappers for automated handling of input and output files between processes, and collection of polypeptide, polynucleotide sequence and features from databases and repositories (e.g., antiSMASH). The sequence comparator 211 can produce an output a file 223 containing sequence data processed according to any aforementioned processes that the sequence comparator 211 is configured to perform. Sequence comparator 211 can be configured to store an output file 223 with a set of files 222 in memory 220, as depicted in step 108 of the example method shown in FIG. 1.

In some embodiments, the feature extractor 212 can be configured to receive or retrieve a file as input that can contain polynucleotide or polypeptide sequences and associated features. In some implementations, the feature extractor 212 extracts features to form, generate, and/or otherwise define a binary feature vector, such as to express, represent, and/or provide indication of the extracted features. In some implementations, the feature extractor 212 can be configured to implement various pattern recognition techniques such as those including parsing, detection, identification, rules, evaluating, generating, and/or defining a set of values associated with the file. The extracted features of the file can include, resemble, or correspond to various data types, such as, for example, streams, files, headers, variable definitions, routines, sub-routines, strings, elements, subtrees, tags, text containing embedded scripts, and/or the like. The extracted features can serve as predictors that can form the input vector of a classifier and/or machine learning model, as described herein. For example, some features may have no impact on the result of the classification, others however may have a direct correlation, and some features may be correlated with and/or interact with other features and in turn be decisive for the outcome. The feature extractor 212 can produce an output a file 223 containing polynucleotide sequences and extracted features according to any aforementioned processes that the feature extractor 212 is configured to perform. Feature extractor 212 can be configured to store an output file 223 with a set of files 222 in memory 220.

In some embodiments, the machine learning model 213 can learn rules from the output of the feature extractor 212. The machine learning model 213 can be, for example, a random forest model, neural network, support vector machine, gradient boosting tree, or another appropriate machine learning model. The process during which the machine learning model 213 learns rules is referred to as “training.” During training the rules learn associations between features extracted from polynucleotide sequence(s) and side information. For the purpose of classification, the side information can be categorical classes; for the purpose of regression the side information can be continuous variables; and for the purpose of ranking the side information can be ordinal relationships. Classes can be higher order categories associated with feature sets of a given sequence. Classes can be, for example, structural, chemical, phenotypic, and/or biosynthetic higher order categories. In a particular implementation, cyclic peptide is a class. In some implementations, structural categories can be heterocyclic rings or lipophilicity; chemical categories can be the amino acid, carboxylic acid or aklyloid substrates; phenotypic categories can be host bioassay or bioactivity assessment; biosynthetic categories can be biosynthesis class The training data set can include, for example, feature sets for polynucleotide or polypeptide sequences and known associated classes.

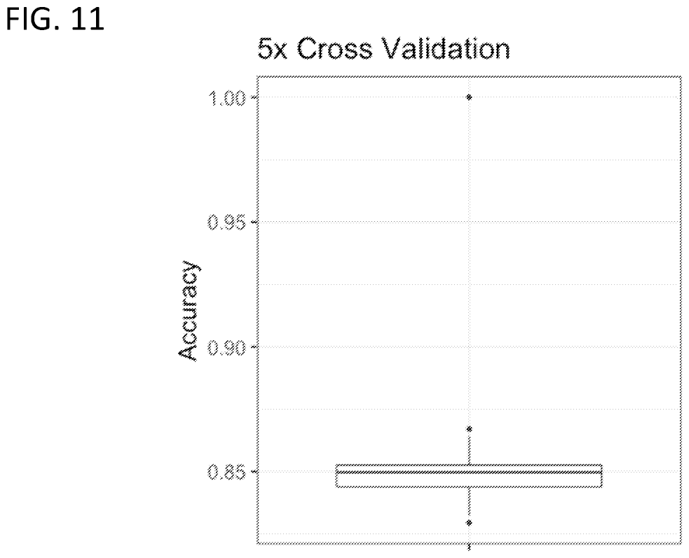

The machine learning model 213 can be configured to execute an analysis to determine the performance accuracy of a trained machine learning model. The analysis to determine the performance accuracy of the trained model is called “validation.” Validation can be, for example, K-fold cross-validation, sensitivity, selectivity analysis and/or the like. The results of the validation can quantify the ability of the trained model to make accurate predictions on polynucleotide or polypeptide sequences with unknown classifications (FIG. 11). Parameters and/or weights associated with the trained and validated machine learning model 213 can be stored in machine learning data 221 within memory 220.

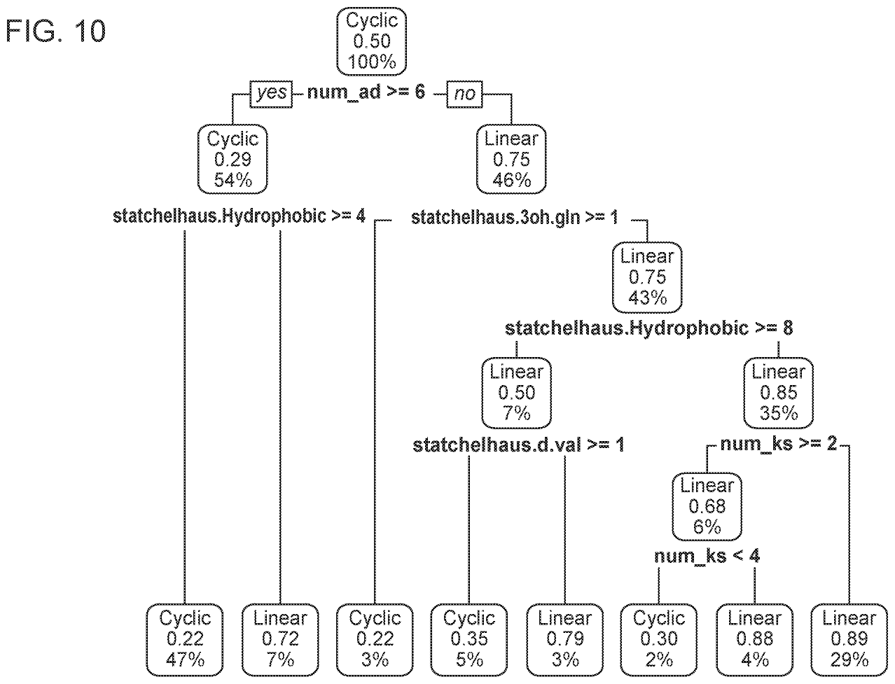

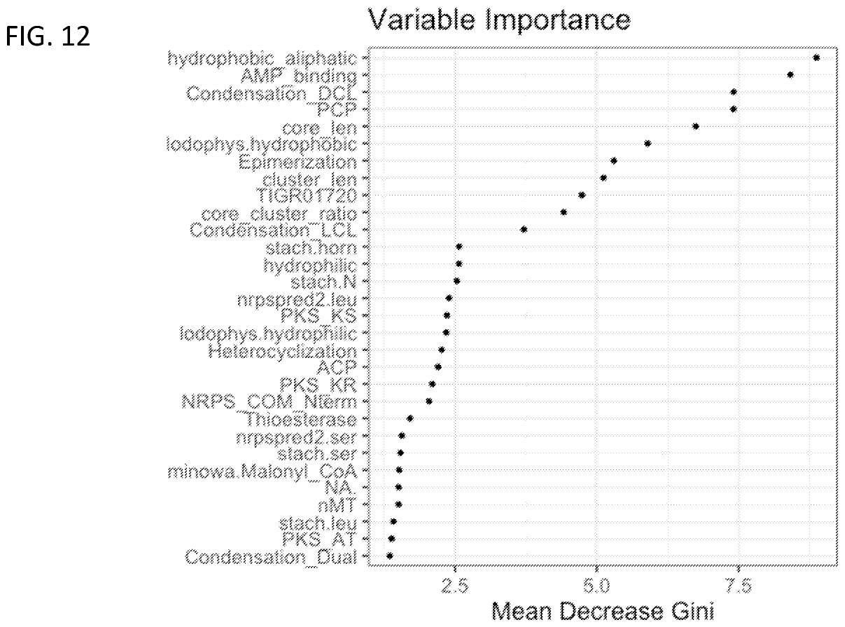

In some embodiments, the gene cluster classifier 214 uses a trained machine learning model to predict associations of classes with features extracted from polynucleotide or polypeptide sequences. Gene cluster classifier 214 can be configured to use the trained machine learning model 213 (using machine learning data 221 within memory 220). Gene cluster feature classifier 214 can be configured to retrieve a file 223 that contains gene sequences and associated feature sets with unknown classes. A data instance from file 223 can include a gene sequence and a set of extracted features. Gene cluster feature classifier 214 can be configured to input one or more data instances into the trained machine learning model 213 and output a new file containing one or more data instances that can be gene sequence(s), a feature set, one or more classes, and statistical information related to the association between the class and the gene sequence and/or feature set. Gene cluster feature classifier 214 can be configured to cluster one or more polynucleotide sequences into groups based on the predicted classes. Gene cluster feature classifier 214 can be configured to rank one or more classes, features, and/or polynucleotide sequences based on the statistical information related to the associations between the class(es) and the gene sequence(s) or feature set(s). For example, FIG. 12 shows a relevance ranking of features used to classify gene clusters as cyclic vs. linear, according to an embodiment. Gene cluster classifier 214 can be configured to output a graphical representation of a polynucleotide sequence classification model such as, for example, decision tree diagrams. For example, FIG. 10 shows a graphical output of a decision tree diagram classifying sequences as cyclic or linear peptides.

In some implementations, a method includes generating annotated gene clusters from long-read genomic sequencing data derived from nucleic acid molecules extracted from an environmental sample. The method includes: 1) extracting nucleic acid molecules from an environmental sample; 2) cloning extracted nucleic acid molecules into DNA vectors; 3) creating a DNA library of the cloned nucleic acid molecules; 4) performing long-read DNA sequencing on the DNA library; 5) partitioning long-read sequencing data into groups using a molecular identifier; 6) performing a first round of read error correction in each group from (5); 7) performing a second round of read error correction in each group from (6); and 8) identifying and annotating gene clusters in the sequences from step (7).

In some implementations, a method includes identifying cyclic peptides from environmental genomic sequence data. The method includes: 1) acquiring at least one nucleotide sequence from a database; 2) acquiring at least one peptide structure(s) corresponding to (1) from a database; 3) extracting and aggregating features from the annotated sequences of (1); 4) labeling the structural data of (2) with structural classifiers; 5) training and validating a machine learning model with the features of (3) and classifiers of (4); 6) use the trained machine learning model of (5) to predict peptide structural classifications from features extracted from sequence data with unknown structural classifications.

In some aspects, the disclosure provides polynucleotides comprising sequences encoding gene clusters, e.g. biosynthetic gene clusters, identified according to the methods or apparatus described herein. Polynucleotides comprising a sequence encoding the gene clusters can be synthesized by any suitable method (see, e.g., Hughes et al. Methods Enzymol 498:277-309 (2011)). Polynucleotides comprising a sequence encoding the gene clusters can be inserted, or cloned, into expression vectors, such as a plasmid-based or viral vector, according to molecular biological techniques and methods known in the art (see, e.g., Green and Sanbrook. Molecular Cloning: A laboratory Manual (Fourth Edition) (2014)).

In some aspects, the disclosure provides expression vectors comprising polynucleotides comprising sequences encoding gene clusters, e.g. biosynthetic gene clusters, The expression vectors may be plasmids, viruses, linear DNA, bacterial artificial chromosomes or yeast artificial chromosomes. Each of the one or more expression vectors may contain one or more promoters suitable for expression of one or more heterologous genes, e.g. a biosynthetic gene cluster identified according to the methods or apparatus described herein, in a model host system. Each expression vector may contain a single coding sequence or multiple coding sequences. Multiple coding sequences may be functionally linked to a single promoter, for example via an internal ribosome entry site, or may be linked to multiple promoters. The expression vectors may also contain additional elements to regulate or increase the transcriptional activity, for example enhancers, polyA sequences, introns, and posttranscriptional stability elements. The expression vectors may also contain one or more selectable markers.

In some implementations, gene clusters, e.g. biosynthetic gene clusters, identified according to the methods or apparatus described herein can be produced in a host cell. In some embodiments, the host cell comprises an expression vector comprising a polynucleotide comprising a sequence encoding a gene cluster, e.g., a biosynthetic gene cluster identified according to the methods or apparatus described herein. Production of the gene clusters in a host cell can be achieved by transfecting, transducing, or otherwise introducing into the host cell an expression vector comprising a polynucleotide with a sequence encoding a gene cluster. Incubating the expression vectors in the host cells allows for the transcription and translation of the coding sequences to recreate the proteins of the gene cluster. These proteins may then produce a desired chemical product which can be isolated from the cells or the media in which the cells are grown.

The host cell may be any cell capable of expressing the coding sequences, e.g. sequences encoding a biosynthetic gene cluster, from the expression vectors. The host cell may be a cell which can be grown and maintained at a high density. For example the host cell may be one which may be grown and maintained in a bioreactor or fermenter. The host cell may be a bacterial cell, a fungal cell, a yeast cell, a plant cell, an insect cell or a mammalian cell.

In some implementations the host cell is bacterial. The bacteria may be a Proteobacteria such as a Caulobacteria, a phototrophic bacteria, a cold adapted bacteria, a Pseudomonads, or a Halophilic bacteria; an Actinobacteria such as Streptomycetes, Norcardia, Mycobacteria, or Coryneform; a Firmicutes bacteria such as a Bacilli, or a lactic acid bacteria. Examples of bacteria which may be used include, but are not limited to: Caulobacter crescentus, Rodhobacter sphaeroides, Pseudoalteromonas haloplanktis, Shewanella sp. strain Ac10, Pseudomonas fluorescens, Pseudomonas putida, Pseudomonas aeruginosa, Halomonas elongata, Chromohalobacter salexigens, Streptomyces lividans, Streptomyces griseus, Nocardia lactamdurans, Mycobacterium smegmatis, Corynebacterium glutamicum, Corynebacterium ammoniagenes, Brevibacterium lactofermentum, Bacillus subtilis, Bacillus brevis, Bacillus megaterium, Bacillus licheniformis, Bacillus amyloliquefaciens, Lactococcus lactis, Lactobacillus plantarum, Lactobacillus casei, Lactobacillus reuteri, and Lactobacillus gasseri.

In some implementations the host cell is a fungal cell. In some cases, the host cell is a yeast cell. Examples of yeast cells include, but are not limited to Saccharomyces cerevisiae, Saccharomyces pombe, Candida albicans, and Cryptococus neoformans. In some cases the host cell may be a filamentous fungi, such as a mold. Examples of molds include, but are not limited to Acremonium, Alternaria, Aspergillus, Cladosporium, Fusarium, Mucor, Penicillium, and Rhizopus. In some cases, the host cell may be an Acremonium cell. In some cases, the host cell may be an Alternaria cell. In some cases, the host cell may be an Aspergillus cell. In some cases, the host cell may be an Cladosporium cell. In some cases, the host cell may be an Fusarium cell. In some cases, the host cell may be a Mucor cell. In some cases, the host cell may be a Penicillium cell. In some cases, the host cell may be a Rhizopus cell.

In some implementations, the host cell is an insect cell, for example, Spodoptera frugiperda (Sf9 or Sf21) cells. In some cases the host cell is a mammalian cell. Examples of mammalian cell lines include HeLa cells, HEK293 cells, B16 melanoma cells, Chinese hamster ovary cells, or HT1080. In some cases, the host cell is a plant cell. In some cases, the host cell may be part of a multicellular host organism.

In some implementations, the host cell is a genetically engineered cell. A genetically engineered cell may contain genetic alterations that enhance expression or reduce degradation of a heterologous protein, e.g. a biosynthetic gene cluster identified by the methods or apparatus described herein. For example, the yeast strain BJ5464 has historically been a strain for expression of heterologous proteins. BJ5464 lacks two vacuolar proteases genes (PEP4 and PRB1), which makes the strain useful for biochemical studies, owing to reduced protein degradation.

In some implementations, a desired chemical can be produced in a host cell comprising an expression vector comprising a polynucleotide with a sequence encoding a gene cluster, e.g. a biosynthetic gene cluster identified according to the methods or apparatus described herein. The host cell expressing a gene cluster, e.g. a biosynthetic gene cluster identified by the methods or apparatus described herein.

In one embodiment, the disclosure provides a non-transitory processor-readable medium storing code representing instructions to be executed by a processor, the instructions comprising code to cause the processor to: obtain long-read sequencing data including a set of reads derived from a sample of genomic deoxyribonucleic acid (DNA), each read from the set of reads includes a polynucleotide sequence; determine a sequence similarity and/or alignment length score between the polynucleotide sequence of each read from the set of reads and the polynucleotide sequence of the remaining reads from the set of reads; generate a network graph representation of the set of reads and the polynucleotide similarity and/or alignment length scores for each read from the set of reads, each node from a set of nodes in the network graph representation represents a single read from the set of reads and each edge from a set of edges between a pair of nodes from the set of nodes represents that the sequence similarity and/or alignment length score between the polynucleotide sequence of a first read from the pair of reads and the polynucleotide sequence of a second read from the pair of reads is above a first threshold; partition each read from the set of reads into a group of reads from a set of groups, the polynucleotide sequence for each read within each group of reads from the set of groups having a higher sequence similarity and/or alignment length score with the polynucleotide sequence for each remaining read in that group of reads than with the polynucleotide sequence for each read within each remaining group of reads from the set of groups; generate a consensus polynucleotide sequence for each group of reads from the set of groups based on the polynucleotide sequence associated with each read from the set of reads in that group; align the consensus polynucleotide sequence for each group of reads from the set of groups with a polynucleotide sequence encoding a polypeptide based on the polynucleotide sequence encoding the polypeptide having a sequence similarity and/or alignment length score with the consensus polynucleotide sequence for that group of reads above a second threshold, the consensus polynucleotide sequence for that group of reads is modified to share a third threshold similarity and/or alignment length with the polynucleotide sequence encoding the polypeptide to produce a modified consensus polynucleotide sequence; classify the modified consensus polynucleotide sequence using a machine learning classifier trained to classify polynucleotide sequences belonging to a biosynthetic gene cluster according to a set of polynucleotide sequence features to identify polynucleotide sequences belonging to one or more biosynthetic gene clusters from long-read sequencing data.

In some implementations, the disclosure provides an apparatus, comprising: a memory; a communicator; and a processor operatively coupled to the memory and the communicator, the processor configured to: obtain long-read sequencing data including a set of reads derived from a sample of genomic deoxyribonucleic acid (DNA), each read from the set of reads includes a polynucleotide sequence; determine a sequence similarity and/or alignment score between the polynucleotide sequence of each read from the set of reads and the polynucleotide sequence of the remaining reads from the set of reads; generate a network graph representation of the set of reads and the polynucleotide similarity and/or alignment length scores for each read from the set of reads, each node from a set of nodes in the network graph representation represents a single read from the set of reads and each edge from a set of edges between a pair of nodes from the set of nodes represents that the sequence similarity and/or alignment length score between a pair of nodes from the set of nodes represents that the sequence similarity and/or alignment length score between the polynucleotide sequence of a first from the pair of reads and the polynucleotide sequence of a second read from the pair of reads is above a first threshold; partition each read from the set of reads into a group of reads from a set of groups, the polynucleotide sequence for each read within each group of reads from the set of groups having a higher sequence similarity and/or alignment length score with the polynucleotide sequence for each remaining read in that group of reads than with the polynucleotide sequence for each read within each remaining group of reads from the set of groups; generate a consensus polynucleotide sequence for each group from the set of groups based on the polynucleotide sequence associated with each read from the set of reads in that group; align the consensus polynucleotide sequence for each group of reads from the set of groups with a polynucleotide sequence encoding a polypeptide based on the polynucleotide sequence encoding the polypeptide having a sequence similarity and/or alignment length score with the consensus polynucleotide sequence for that group of reads above a second threshold level, the consensus polynucleotide sequence for that group of reads is modified to share a third threshold sequence similarity and/or alignment length with the polynucleotide sequence encoding the polypeptide to produce a modified consensus polynucleotide sequence; classify the modified consensus polynucleotide sequence using a machine learning classifier trained to classify polynucleotide sequences belonging to a biosynthetic gene cluster according to a set of polynucleotide sequence features; and output a report identifying polynucleotide sequences belonging to a biosynthetic gene cluster. In some implementations, the first threshold level is at least 80%, at least 85%, at least 90%, at least 95%, at least 96%, at least 97%, at least 98%, at least 99%, or 100% sequence similarity and/or alignment length. In some implementations, the first threshold is at least 80%, at least 85%, at least 90%, at least 95%, at least 98% or at least 99%. In some implementations, the second threshold level is at least 80%, at least 85%, at least 90%, at least 95%, at least 98% or at least 99%.

In some implementations, the apparatus has a memory, a processor, and a communicator for predicting classifiers of extracted features from polynucleotide sequences. The apparatus comprises: 1) a memory configured for storing machine learning data; 2) a memory configured for storing polynucleotide sequences, extracted features, and classifiers; 3) a processor configured for assembling and error correcting polynucleotide sequences from long-read sequencing data; 4) a processor configured for extracting features from a plurality of polynucleotide sequences; 5) a processor configured for training and validating at least one machine learning model with a plurality of polynucleotide sequences, extracted features, and/or classifiers; 6) a processor configured for predicting classifiers and associated statistical data for extracted feature sets of polynucleotide sequences with unknown classifiers; 7) a processor configured for ranking classifiers associated with polynucleotide sequences and extracted feature sets from (6).

### EXAMPLES

### Example 1. Informatics Pipeline Concept

The informatics pipeline described by the methods and apparatus described herein address important issues related to obtaining high quality genomic fragments from long-read sequencing data from which biosynthetic gene clusters can be identified (FIGS. 1-4). The informatics pipeline overcomes major technical hurdles associated with processing long-read sequencing data from sequenced genomic libraries, such as libraries based on sequences cloned into cosmids. For example, one technical challenge is obtaining multiple reads from the same exact cosmid with no incorrect mappings. The informatics pipeline is designed to take advantage of libraries with specific nucleotide lengths. For example, read lengths containing 40 kb sequences can use the read itself as a Universal Molecular Identifier based on sequence similarity and/or alignment length. The informatics pipeline can properly identify identical full length reads of 40 kb, or a another defined length, and group the reads by requiring a threshold similarity and/or alignment length over the full length of the sequence. This insight allows partitioning of all reads in a sequencing run, followed by using the grouped reads for error correction by generating a highly accurate consensus sequences that shares about 99% identity to the actual sequence in the library.

Another technical hurdle the informatics pipeline overcomes is identifying open reading frames, or coding regions of the sequence that can be translated to functional polypeptides. To solve this problem, the informatics pipeline uses a frameshift-aware aligner that aligns the polynucleotide consensus sequence derived from the long-read sequencing data with a polynucleotide sequence that encodes a known polypeptide. The aligner is able to identify the gaps, stop codons, frameshift mutations, or nucleotide substitutions that would prevent translation of the polypeptide. in the consensus polynucleotide sequence. When the translation of DNA-to-protein hits a gap or a stop codon, for example, it is allowed to look in the +1 or −1 reading frame to confirm whether the protein aligns. If so, the alignment continues. By looking for strong alignments and noting where the frameshifts occur, the consensus polynucleotide sequence derived from the long-read sequencing data can be corrected such that the polypeptide is properly encoded. And because small insertions/deletions are the most common class of errors in sequencing data from long-read sequencing methods, this is, surprisingly, a very effective method.

The combined strategy of the using sequencing similarity and/or alignment length to first form groups of reads using sequence, determining an associated consensus sequence for reads with each group, followed by frameshift aware sequence correction has, surprisingly, allowed efficient processing of long-read sequencing data and accurate identification of gene cluster sequences that created an order of magnitude improvement over other state of the art methods.

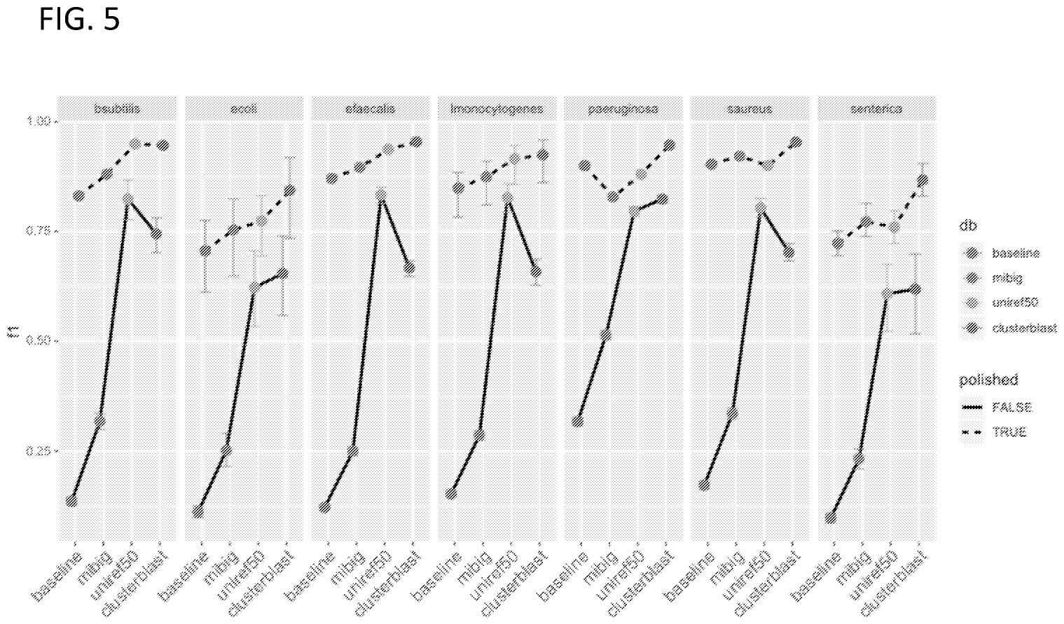

Example 2. A published dataset of long-read nanopore sequencing data derived from seven bacterial species in a mock metagenome was used to assess frameshift-aware error correction (FIG. 5). Agreement between the predicted results of the example method and a reference database was quantified using an F₁ score, a measurement of the precision and recall of a machine learning model. For each genome F₁ scores greater than 0.9 were obtained, indicating high-agreement between the protein-or protein-domain content of reference data compared with the genome sequencing data processed using frameshift-aware error correction, as described in the example method of FIG. 1. F₁ scores obtained from different conditions where sequence data were processed with or without racon (polished=true/false) and using different reference protein databases for error correction (in size order: no database, mibig, clusterblast, or uniref50 protein datasets). For each combination of polishing and reference dataset, the open reading frames were predicted with the prodigal ORF-caller and protein domain content was assessed based on the PFAM subroutines contained in antiSMASH. Proteins and domains were considered to be correctly identified if they shared 99% identity over at least 75% percent of sequence length to the gold-standard reference sequence. F₁ scores were calculated by tabulating true positive, true negative and false positive protein and domain content to the reference according to the criteria above and using these to calculate the precision and recall of the different experimental condition. The harmonic mean of precision and recall is the F₁ score. Taken together, the use of the two-step process of polishing plus error correction can result in high agreement of experimental data with reference genomes.

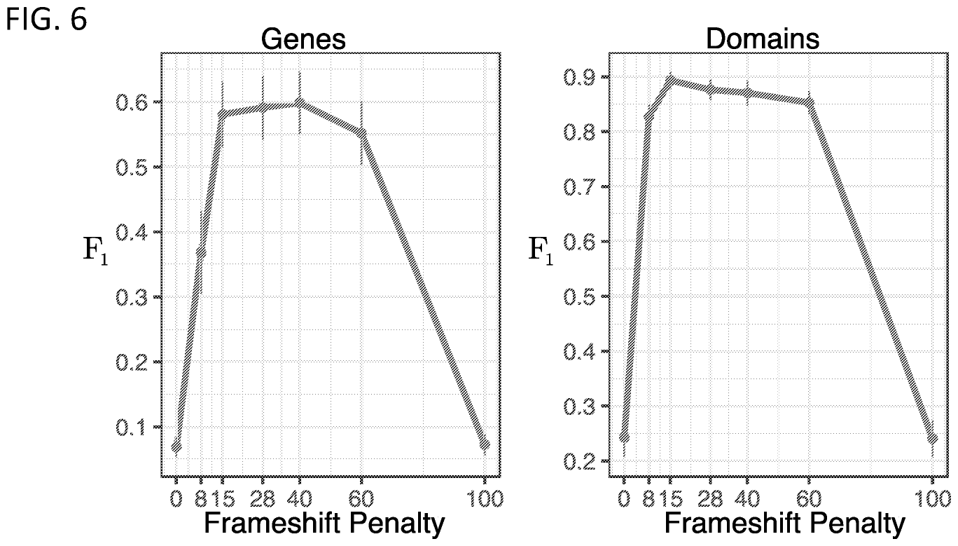

Example 3. A set of metagenomic-derived gene clusters and the clusterblast protein reference dataset was used to evaluate the effect of varying the frameshift penalty used by the frameshift-aware protein to DNA on the recovery of protein features when measuring either protein (Genes) or protein-domain content (Domains) (FIG. 6). Agreement between the clusters processed as described in the example method of FIG. 1 and the reference dataset was quantified using an F₁ score. For both Genes and Domains, a frameshift score of 15 generated the highest F₁ scores with strong agreement (F₁>0.9) between a gold-standard reference (sequenced by either PacBio or PacBio plus ILMN) and reads obtained by nanopore sequencing.

Example 4. The effect of read coverage on F₁ score using the example method of FIG. 1 was assessed using the datasets described above, and frameshift-aware error correction and frameshift penalty settings identified previously (FIG. 7). Using either protein (Genes) or domain-level (Domains) annotations, F₁ scores greater than 0.7 were obtained using as few as 10 reads. The results show that the F₁ scores generally increase with more reads and that the lowest number of reads can result in valuable information that can be extracted from genomic regions.

Example 5. The examples above indicate the ability to obtain high quality, annotated consensus sequences from an adequate number of long sequence reads covering the same unique molecular identifier. From the apparatus depicted in FIG. 2 and described herein, corrected sequences from a mixture of bacterial populations can be obtained. Long read sequencing produces noisy, error-prone reads and the difficulty of obtaining sufficient reads for error correction lies in the difficulty of assigning a read from a mixed population to its source material: sequences from comparable genomes in a metagenomic pool too similar to be distinguished. However, cloning metagenomic fragments prior to long read sequencing resolves this ambiguity. Cosmid sequences that are linearized along the backbone of the cloning vector can be sequenced as a single long read. Furthermore, we expect to sequence multiple copies of the cosmid, within some error tolerance and spatial resolution. Therefore, the full length sequence itself is a unique identifier by which the process groups reads from the same source cosmid. The process involves an all-against-all comparison of sequences in an experiment and assesses their pairwise similarity using a noise-tolerant read aligner, such as minimap2. The process filters the outputs of minimap2 to keep only those read pairs above some length and similarity threshold. In one instance, this threshold is 90%. The output of the all-against-all comparison is a table of pairwise similarities where a unique identifier is assigned to each read. The process ingests this table in the form of an edge list and converts it to a data structure known as an undirected graph using the igraph package. In this graph, nodes represent the long read sequences and edges represent similarity scores and/or alignment lengths between nodes that exceed the threshold. Nodes are then grouped into reads with a common molecular origin. For example, in FIG. 3B, node N001 can represent a first read, node N002 can represent a second read, and edge E001 can represent the sequence similarity and/or alignment length between the first and second reads. The process can use, for example, the Louvain algorithm to partition nodes (reads) into a module set (group). For example, as shown in FIG. 3B, node N001 and node N002 are within module (or group) 1. Other nodes are grouped within other modules or groups (e.g., module 2 and module 3). Once the modules are detected, the process writes out node identifiers and sequence reads from the original dataset to a FASTA file. Each sequence FASTA file contains subpools of noisy long reads that are sufficiently similar and are inferred as arising from a common source, their sequence similarity acts as a unique inherent identifier. The process uses the subpools stored as sequences in in FASTA files, to generate consensus sequences to be polished and annotated. The process is outlined in (FIG. 3A and FIG. 3B).

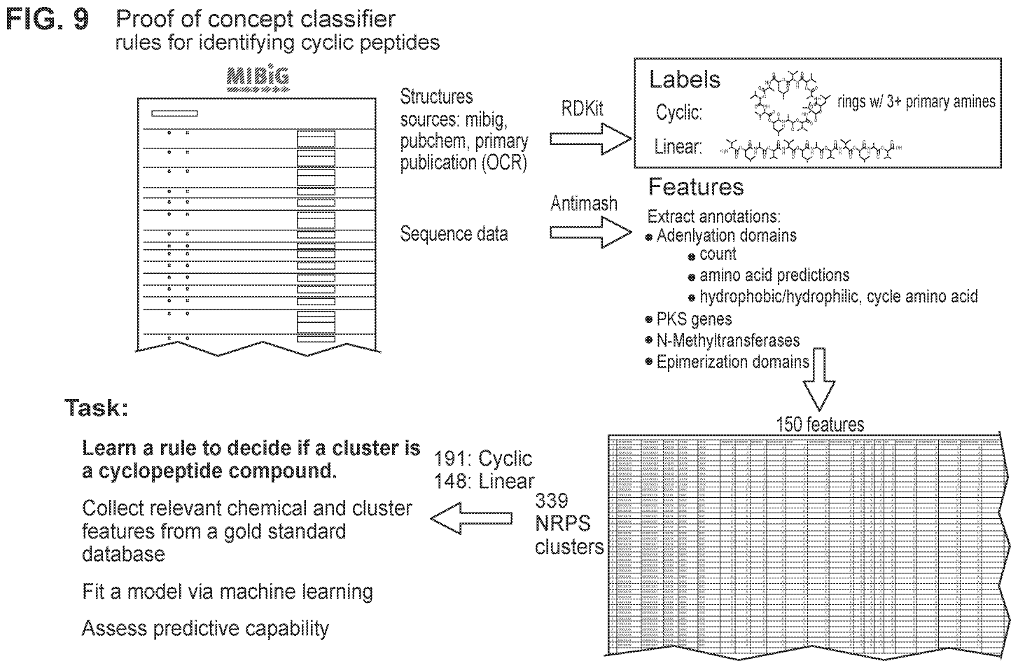

The above examples indicate the ability to generate high quality genome assemblies from metagenomic sources and use those assemblies to extract biochemically relevant features such as proteins and enzymatic protein-domain content. From this set of features, machine learning models (i.e., classifiers) can be built for predicting chemical and/or biochemical aspects of gene clusters. To demonstrate this, a proof of concept classifier was created (FIG. 9). The classifier was built by extracting genomic features from a known dataset (the MIBiG dataset) and providing labels to classify the biochemistry encoded by the clusters. The labels categorized the gene clusters as either encoding a linear or cyclic peptide. A random forest model was trained and validated using the labeled dataset, as described herein. The random forest model was validated using 5-fold cross validation, i.e., the model's accuracy was determined using a random subset of the data that was not used for training the model. The accuracy of the model was greater than 85% (FIG. 11). Furthermore, the relative importance of features used to generate these predictions can be assessed (FIG. 12).

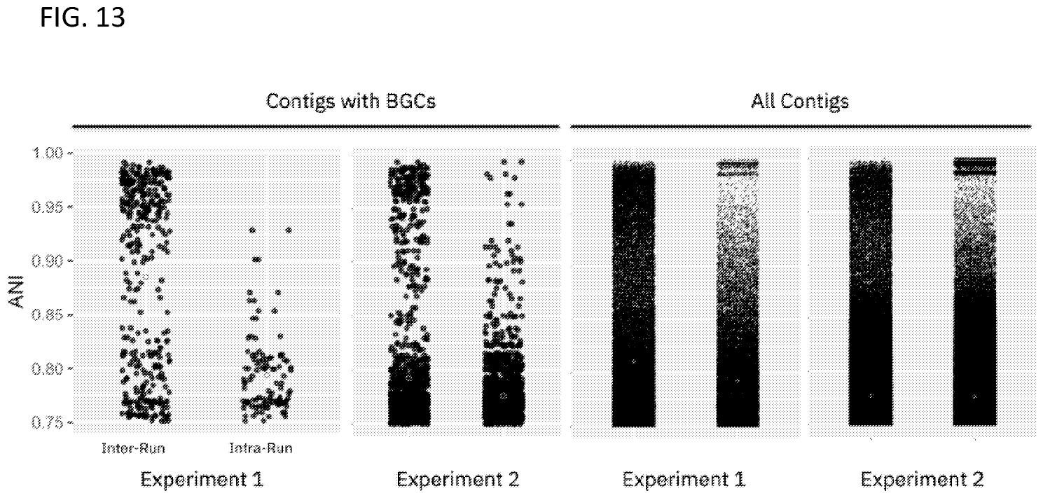

Example 6. A critical component required for the first error correction step of long-read sequences is partitioning the reads into groups, also called modules, such that each read in the group represents an error-prone sequence read of the same sequence. These reads are grouped together and used to obtain a consensus sequence for the cosmid. The consensus sequence represents an error-corrected version of the read sequence. If we are too conservative in thresholding edges or partitioning the graph, then we may end up with multiple consensus reads for the same cosmid (false positives). Conversely, groups that are too large, or over-inclusive of reads, may represent distinct cosmids and ultimately hybrid consensus sequences (false negatives) each at reduced read coverage. The graph partitioning algorithm finding false positives is a simple matter of looking for duplicate sequences (within some identity threshold) among the consensus sequences. To find a threshold that will reveal false positive, two experiments where the same source DNA was independently resequenced (FIG. 13). In Experiment 1, library L1891 was sequenced three times from the same source material. In Experiment 2, a row pool from L1063 containing well L1063-241 was sequenced, and L1063-241 was also sequenced independently. All sequencing was performed using nanopore sequencing (Oxford Nanopore Sequencing). FastANI was used to compare the inter- and intra-run contig identities for those two experiments, the results of which can be found in FIG. 13. In these experiments high ANI values (85-95%) represent the same sequence. In the intra-run experiments these are contigs that we would expect to be identical clusters that should not have remained split during graph partitioning (false duplicate). The number of these reads relative to the total is on the order of 3-5%. Less than 3% of the contigs in an experiment are present in the intra-well comparison suggesting an upper bound of false duplicate at about 3%. Post-Excavator Contigs (All Contigs) or contigs that contain BGCs were analyzed using Fast ANI.

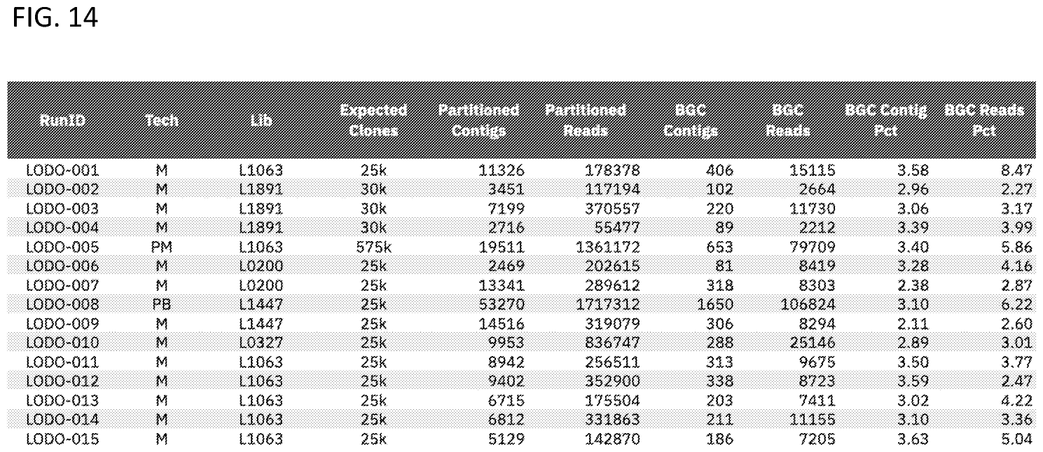

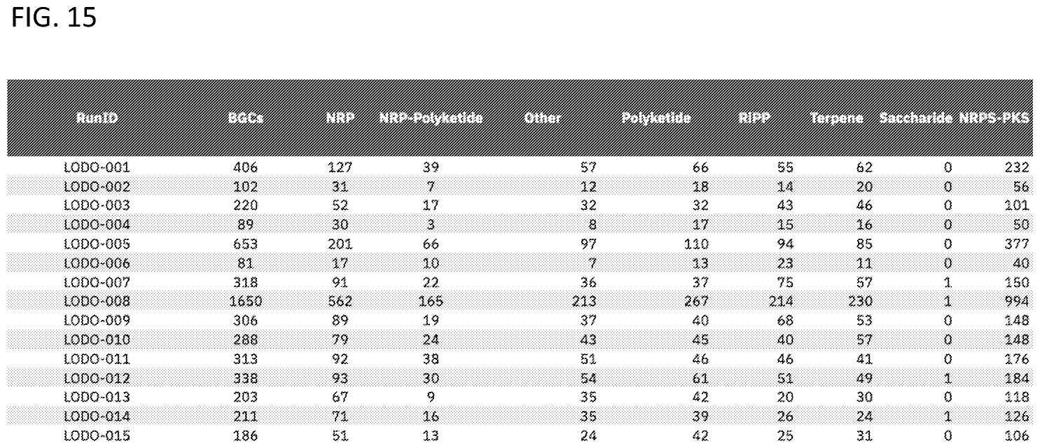

Example 7. FIG. 14 summarizes data from experiments that sequenced wells from which clones had previously been recovered. The bioinformatic pipeline was run on a set of experimental datasets (FIG. 14, RunID column). All reads were used to create and polish the contigs but only the contigs of 30 kb or greater were retained for the statistical analysis. In FIG. 14, Lib refers to the reference library; Tech column refers to the long-read sequencing technology used to generate the dataset (PM: Promethion; M: Minion; PB: PacBio Sequel II); Expected Clones is the pool size of the eDNA clones; Expected reads is the total number of reads identified by the informatics pipeline; Paritioned Contigs is the number of contigs generated by the informatics pipeline from the identified reads; Partitioned Reads refers to the number of reads grouped in the informatics pipeline; BGC contigs and BGC reads are the number of contigs and reads containing a biosynthetic gene cluster, respectively; BGC Contig Pct and BGC Reads Pct list the percent of contigs of total contigs containing a BGC and the percent of reads of total reads containing a BGC, respectively. FIG. 15 shows the total number of BGCs and the number of each class of BGC, such as a non-ribosomal peptide synthetase/polyketide synthase (NRPS-PKS) cluster. In addition to the NRPS-PKS BGCs identified by this method, a large number of BGCs are identified in each of the sequenced samples as indicated in FIG. 15 suggesing the informatics pipeline can identify novel gene clusters from long-read sequencing data.

It should be understood that the disclosed embodiments are not representative of all claimed innovations. As such, certain aspects of the disclosure have not been discussed herein. That alternate embodiments may not have been presented for a specific portion of the innovations or that further undescribed alternate embodiments may be available for a portion is not to be considered a disclaimer of those alternate embodiments. Thus, it is to be understood that other embodiments can be utilized, and functional, logical, operational, organizational, structural and/or topological modifications may be made without departing from the scope of the disclosure. As such, all examples and/or embodiments are deemed to be non-limiting throughout this disclosure.

Some embodiments described herein relate to methods. It should be understood that such methods can be computer implemented methods (e.g., instructions stored in memory and executed on processors). Where methods described above indicate certain events occurring in certain order, the ordering of certain events can be modified. Additionally, certain of the events can be performed repeatedly, concurrently in a parallel process when possible, as well as performed sequentially as described above. Furthermore, certain embodiments can omit one or more described events.

All definitions, as defined and used herein, should be understood to control over dictionary definitions, definitions in documents incorporated by reference, and/or ordinary meanings of the defined terms.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

Some embodiments and/or methods described herein can be performed by software (executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including C, C++, Java™, Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments can be implemented using Python, Java, JavaScript, C++, and/or other programming languages and software development tools. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

The drawings primarily are for illustrative purposes and are not intended to limit the scope of the subject matter described herein. The drawings are not necessarily to scale; in some instances, various aspects of the subject matter disclosed herein can be shown exaggerated or enlarged in the drawings to facilitate an understanding of different features. In the drawings, like reference characters generally refer to like features (e.g., functionally similar and/or structurally similar elements).

The acts performed as part of a disclosed method(s) can be ordered in any suitable way. Accordingly, embodiments can be constructed in which processes or steps are executed in an order different than illustrated, which can include performing some steps or processes simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of” “only one of” or “exactly one of” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

We claim:

1. A method of identifying biosynthetic gene clusters from long-read sequencing data, comprising:
obtaining long-read sequencing data including a set of reads derived from a sample of genomic deoxyribonucleic acid (DNA), each read from the set of reads includes a polynucleotide sequence;
partitioning each read from the set of reads into a group of reads from a set of groups, the polynucleotide sequence for each read within each group of reads from the set of groups having a higher alignment length with the polynucleotide sequence for each remaining read in that group of reads than with the polynucleotide sequence for each read within each remaining group of reads from the set of groups;
performing a first read error correction for each group of reads from the set of groups by generating a consensus sequence associated with that group of reads;
performing a second read error correction for each group of reads from the set of groups by aligning the consensus sequence for that group of reads with a polynucleotide sequence encoding a polypeptide, wherein the consensus sequence is modified to encode the polypeptide, thereby generating a modified consensus polynucleotide sequence;
classifying the modified consensus polynucleotide sequence for each group of reads from the set of groups using a machine learning classifier trained to classify polynucleotide sequences belonging to a biosynthetic gene cluster according to a set of polynucleotide sequence features, the classifying including identifying the modified consensus sequence for each group of reads from the set of groups as having the features of the biosynthetic gene cluster; and
expressing the modified consensus polynucleotide sequence in a host cell based on identifying the modified consensus sequence as having the features of the biosynthetic gene cluster.

2. The method of claim 1, wherein the polynucleotide sequence of each read within each group of reads has an alignment length of at least 90%, at least 95%, at least 98%, or at least 99% with the polynucleotide sequence of each remaining read in that group of reads.

3. The method of claim 1, wherein the long-read sequencing data is obtained from a database.

4. The method of claim 1, wherein the long-read sequencing data is obtained by sequencing a sample of genomic DNA using a long-read sequencing method.

5. The method of claim 4, wherein the sample of genomic DNA is digested into fragments, and wherein the fragments are cloned into a genomic DNA library prior to sequencing.

6. The method of claim 5, wherein the genomic DNA library includes cosmid vectors.

7. The method of claim 1, wherein the machine learning classifier is trained using a set of training data including features extracted from polynucleotide sequences encoding biosynthetic gene clusters and associated classifications, wherein the set of training data is retrieved from a database.

8. The method of claim 7, wherein the features are selected from open-reading-frames, protein-domain content of open reading frames, promoter binding sites, substrate specificity prediction of enzymatic open reading frames, active site prediction of enzymatic open reading frames.

9. The method of claim 7, wherein the classifications are selected from structural, chemical, phenotypic, or biosynthetic higher order categories.

10. The method of claim 7, wherein the machine learning classifier includes at least one of a neural network, a decision tree, a random forest, a support vector machine, a gradient boosting tree, a Bayesian network, or a genetic algorithm.

11. A non-transitory processor-readable medium storing code representing instructions to be executed by a processor, the instructions comprising code to cause the processor to:
obtain long-read sequencing data including a set of reads derived from a sample of genomic deoxyribonucleic acid (DNA), each read from the set of reads includes a polynucleotide sequence;
determine an alignment length between the polynucleotide sequence of each read from the set of reads and the polynucleotide sequence of the remaining reads from the set of reads;
generate a network graph representation of the set of reads and the alignment length for each read from the set of reads, each node from a set of nodes in the network graph representation represents a single read from the set of reads and each edge from a set of edges between a pair of nodes from the set of nodes represents that the alignment length between the polynucleotide sequence of a first read from a pair of reads and the polynucleotide sequence of a second read from the pair of reads is above an alignment length threshold;
partition each read from the set of reads into a group of reads from a set of groups, the polynucleotide sequence for each read within each group of reads from the set of groups having a higher alignment length with the polynucleotide sequence for each remaining read in that group of reads than with the polynucleotide sequence for each read within each remaining group of reads from the set of groups;
generate a consensus polynucleotide sequence for each group of reads from the set of groups based on the polynucleotide sequence associated with each read from the set of reads in that group;
align the consensus polynucleotide sequence for each group of reads from the set of groups with a polynucleotide sequence encoding a polypeptide, wherein the consensus polynucleotide sequence for that group of reads is modified to encode the polypeptide to produce a modified consensus polynucleotide sequence;
classify the modified consensus polynucleotide sequence for each group of reads from the set of groups using a machine learning classifier trained to classify polynucleotide sequences belonging to a biosynthetic gene cluster according to a set of polynucleotide sequence features to identify polynucleotide sequences belonging to one or more biosynthetic gene clusters from long-read sequencing data.

12. The non-transitory processor-readable medium of claim 11, wherein the alignment length threshold is at least 90%, at least 95%, at least 98%, at least 99%, or 100% alignment length.

13. The non-transitory processor-readable medium of claim 11, wherein the polynucleotide sequence encoding a polypeptide is from a database of biosynthetic gene clusters.

14. An apparatus, comprising:
a memory;
a communicator; and
a processor operatively coupled to the memory and the communicator, the processor configured to:
obtain long-read sequencing data including a set of reads derived from a sample of genomic deoxyribonucleic acid (DNA), each read from the set of reads includes a polynucleotide sequence;
determine an alignment length between the polynucleotide sequence of each read from the set of reads and the polynucleotide sequence of the remaining reads from the set of reads;
generate a network graph representation of the set of reads and the polynucleotide alignment lengths for each read from the set of reads, each node from a set of nodes in the network graph representation represents a single read from the set of reads and each edge from a set of edges between a pair of nodes from the set of nodes represents an alignment length between the polynucleotide sequence of a first read from a pair of reads and the polynucleotide sequence of a second read from the pair of reads is above an alignment length threshold of the length of either of the reads;
partition each read from the set of reads into a group of reads from a set of groups, the polynucleotide sequence for each read within each group of reads from the set of groups having a higher alignment length with the polynucleotide sequence for each remaining read in that group of reads than with the polynucleotide sequence for each read within each remaining group of reads from the set of groups;
generate a consensus polynucleotide sequence for each group from the set of groups based on the polynucleotide sequence associated with each read from the set of reads in that group;
align the consensus polynucleotide sequence for each group of reads from the set of groups with a polynucleotide sequence encoding a polypeptide based on the polynucleotide sequence encoding the polypeptide, wherein the consensus polynucleotide sequence for that group of reads is modified to encode the polypeptide, thereby producing a modified consensus polynucleotide sequence;
classify the modified consensus polynucleotide sequence for each group of reads from the set of groups using a machine learning classifier trained to classify polynucleotide sequences belonging to a biosynthetic gene cluster according to a set of polynucleotide sequence features; and
output a report identifying polynucleotide sequences belonging to the biosynthetic gene cluster.

15. The apparatus of claim 14, wherein the alignment length threshold is at least 90%, at least 95%, at least 98%, at least 99%, or 100% alignment length.

16. The apparatus of claim 14, wherein the polynucleotide sequence encoding a polypeptide is from a database of biosynthetic gene clusters.
